(19) United States
(12) Patent Application Publication
(10) Pub. No.: US 2001/0049837 A1
(43) Pub. Date: Dec. 13, 2001

(54) HELMET

(76) Inventor: GORDON SLACK, NORTH LINCOLNSHIRE (GB)

Correspondence Address: HICKMAN STEPHENS COLEMAN & HUGHES, LLP, PO BOX 52037, PALO ALTO, CA, 94303, US

(21) Appl. No.: 09/556,904

(22) Filed: Apr. 20, 2000

(30) Foreign Application Priority Data
Apr. 23, 1999 (GB) 9909340.3

(51) Int. Cl. F41H 1/04; A42B 3/00; A42B 3/30; A42B 1/00; A61F 9/00

(52) U.S. Cl. 2/6.2

(57) ABSTRACT

A helmet comprising

a helmet shell;

a breathing apparatus face mask and a visor, for breathing apparatus and the visor being connected to the helmet shell;

an augmented reality viewer Positioned within the helmet shell and adapted to receive a video signal and to display the signal as a video image, the video image being arranged such that it can be viewed by a wearer of the helmet;

the helmet further comprising a power socket adapted to be connected to an external source of power for the augmented reality viewer.

The present invention relates to a helmet. More particularly, but not exclusively, the present invention relates to a helmet for use by firefighters and search and rescue teams in smoke or thick fog.

It is extremely difficult to find accident or fire victims in conditions of poor visibility. This problem is particularly well know to fire fighters who are required to search for and rescue victims of smoke inhalation in smoke filled environments such as buildings, ships, oil rigs, tunnels etc. It is necessary to find such victims as quickly as possible. Even slight delays can have important consequences for the future health of the victims.

In an attempt to overcome this problem fire fighters and search and rescue crews often use hand held thermal imaging cameras. However, when using such a camera it is necessary for the operator to hold the camera up to his/her breathing apparatus face mask, and look into the camera monitor and then at his/her surroundings. This can make it difficult for the operator to interpret the image produced by the camera monitor. Mobility is also made difficult because his/her hands are not free. Also, whilst using the camera the operator may suffer from tunnel vision.

Helmets having integrated thermal imaging cameras are also known. Such helmet mounted systems comprise a helmet shell adapted to receive the head of a wearer. Located within the helmet shell is a thermal imaging camera, electronics to process the image received by the camera and an augmented reality viewer to display the image. These components are powered by a power source also located within the helmet shell. Such integrated systems are relatively heavy.

Accordingly, in a first aspect the present invention provides a helmet comprising

a helmet shell;

a breathing apparatus face mask for connection to a breathing apparatus and a visor, the breathing apparatus face mask and the visor being connected to the helmet shell;

an augmented reality viewer positioned within the helmet shell and adapted to receive a video signal and to display the signal as a video image, the video image being arranged such that it can be viewed by a wearer of the helmet;

the helmet further comprising a power socket adapted to be connected to an external power source for the augmented reality viewer.

As with the integrated system a wearer of the helmet according to the invention can view the video image from the camera without having to hold the camera to the helmet, so overcoming the problems of lack of mobility and tunnel vision of the wearer. However, unlike the integrated system the helmet according to the invention can be used with a power source located separately from the helmet, for example around the waist or on the back of the wearer. This reduces the weight of the helmet, so enabling it to be worn for longer periods.

Preferably, the video image displayed by the augmented reality viewer is arranged such that it can be viewed by the wearer of the helmet whilst looking through the visor. The video image therefore overlies the image seen by the wearer though the visor which greatly facilitates interpretation of the video image.

Preferably, the augmented reality viewer is located behind the breathing apparatus face mask. This has the advantage that no matter how dirty and obscured the visor becomes the video image is not affected.

Preferably the helmet further comprises a video socket adapted to receive a video signal from an external source for display by the augmented reality viewer. Such a helmet is able to receive a video signal from an external video camera and display the resulting video image so that it can be seen directly by the wearer of the helmet. As the helmet does not include a camera its weight is further reduced. Also, because the helmet can be used with an external thermal imaging camera it can be purchased by fire brigades which have already invested large sums in such cameras.

Preferably the external video source comprises a thermal imaging camera.

Preferably the helmet further comprises a thermal imaging sensor, the thermal imaging sensor being adapted to be connected to an external image interpretation circuit, the image interpretation circuit being adapted to generate a video signal in response to the signal received from the thermal imaging sensor; and, the video socket being adapted to receive the video signal generated by the external image interpretation circuit for display by the augmented reality viewer.

In such an arrangement a relatively light thermal imaging sensor is located in the helmet whilst the heavier image interpretation circuit is located external to the helmet, for example around the waist or on the back of the wearer. This enables hands free operation of the thermal imaging sensor without significant increase the weight of the helmet.

Preferably the helmet further comprises a thermal imaging sensor, the thermal imaging sensor being connected to an integrated image interpretation circuit;

the image interpretation circuit being adapted to generate a video signal in response to the signal received from the thermal imaging sensor; and,

the augmented reality viewer being adapted to receive the video signal from the image interpretation circuit for display.

In such an arrangement a relatively light thermal imaging sensor and integrated image interpretation circuit can be located within the helmet so enabling hands free operation of the thermal imaging sensor.

In a further aspect of the invention there is provided a viewing system for mounting on a breathing apparatus face mask, the viewing system comprising

an augmented reality viewer adapted to receive a video signal and to display the video signal as a video image, the video image being arranged such that it can be viewed by a wearer of the face mask; and,

a power socket adapted to be connected to an external source of power for the augmented reality viewer.

Preferably, the viewing system according to the invention further comprises a thermal imaging sensor and integrated image interpretation circuit, the image interpretation circuit being adapted to generate a video signal in response to the signal received from the thermal imaging sensor; the augmented reality viewer being adapted to receive the video image generated by the image interpretation circuit for display.

Preferably, the viewing system according to the invention further comprises a thermal imaging sensor adapted to be connected to an external image interpretation circuit, the external image interpretation circuit being adapted to generate a video signal in response to the signal received from the thermal imaging sensor; the augmented reality viewer being adapted to receive the video image generated by the image interpretation circuit for display.

The present invention will now be described by way of example only, and not in any limitative sense, with reference to the accompanying drawings of which:

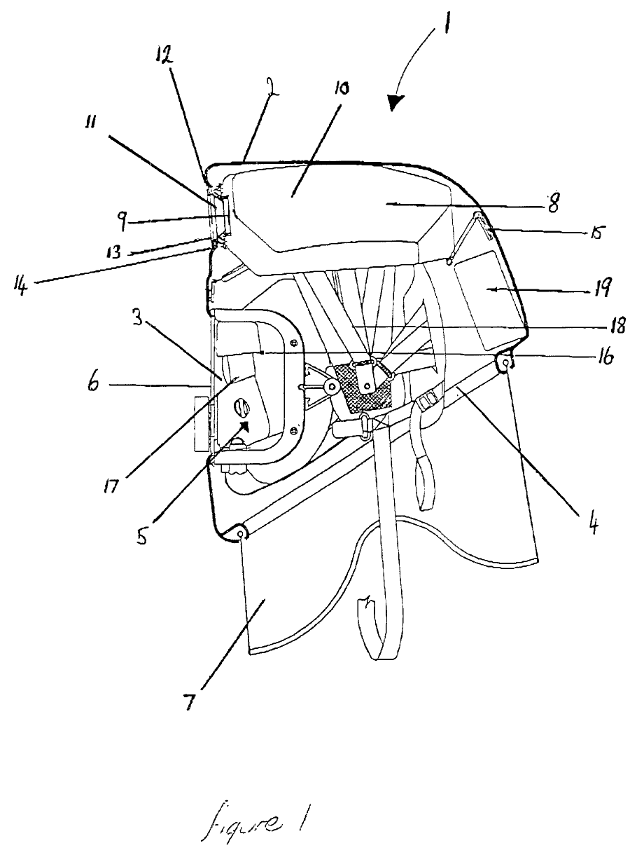

FIG. 1 is a cross sectional view of a known helmet including a thermal imaging camera, an augmented reality viewer and a power supply;

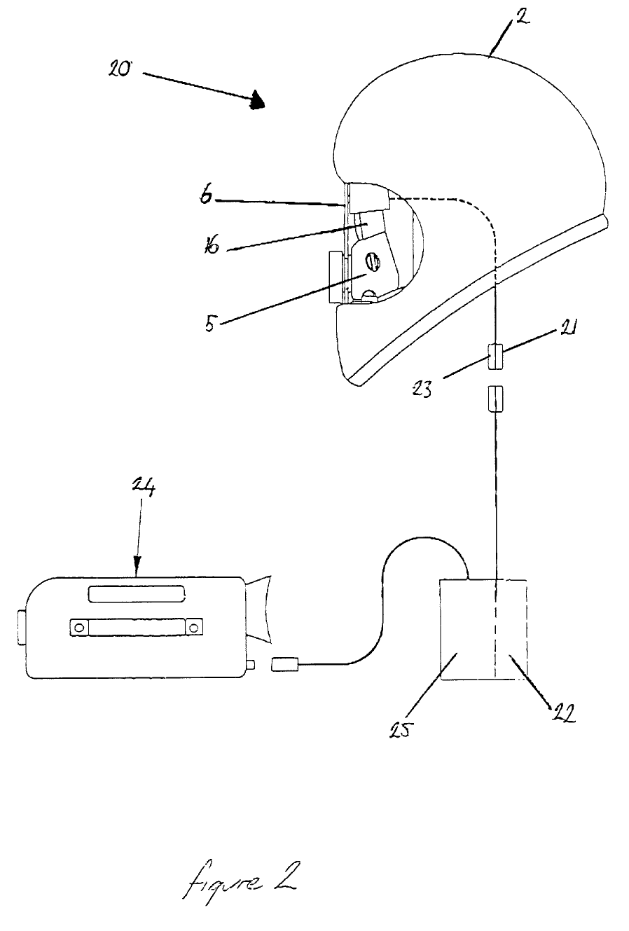

FIG. 2 is a schematic view of a helmet according to a first embodiment of the invention;

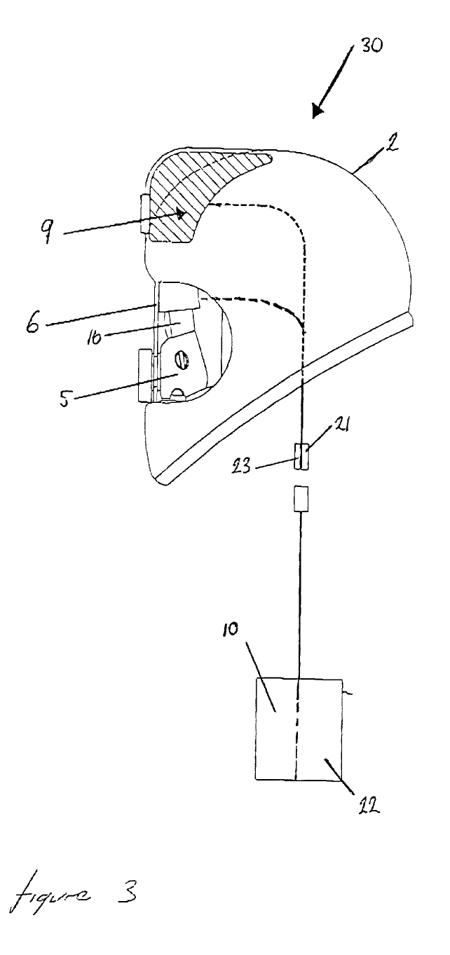

FIG. 3 is a schematic view of a helmet according to a second embodiment of the invention;

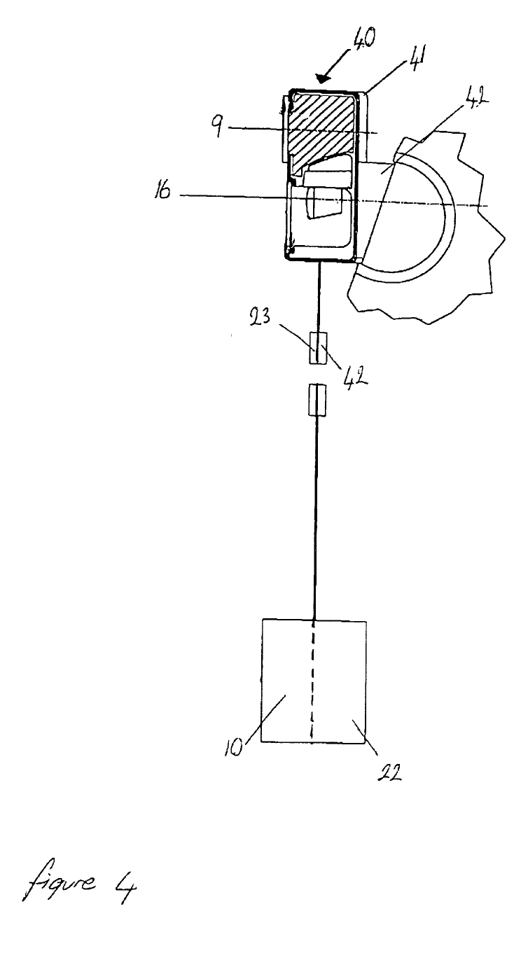

FIG. 4 is a schematic view of a viewing system according to a third embodiment of the invention.

Shown in FIG. 1 is a known helmet 1 used by fire fighters and search and rescue teams. The helmet 1 comprises a helmet shell 2 having a visor aperture 3 and an entrance aperture 4. Sealingly attached to the edge of the visor aperture 3 is a breathing apparatus face mask 5 for connection to a breathing apparatus and a transparent visor 6. Connected around the entrance aperture 4 is a flame resistant neck skirt 7, preferably made of nomex. The shell 2 is typically a glass fibre/Kevlar shell bonded with a fire retardant resin. The visor 6 is typically a polycarbonate.

Located within the helmet shell 2 is a thermal imaging camera 8 comprising a thermal imaging sensor 9 and an image interpretation circuit 10. The lens 11 of the thermal imaging camera 8 extends through an aperture 12 located above the visor 6. The thermal imaging camera 8 is arranged to point in the same direction as the wearer of the helmet 1. It is held in the correct position by a spigotted retainer 13 and ring 14 located at the front and a spring hook 15 located at the rear of the helmet 1.

Located inside the visor 6 of the helmet 1 is an augmented reality viewer 16. The augmented reality viewer 16 is adapted to receive a video signal from the thermal imaging camera 8 and to display this on a transparent member 17. The transparent member 17 is arranged to be at the eye level of the wearer of the helmet 1.

In use the wearer inserts his/her head through the neck skirt 7 and entrance aperture 4 and into the helmet shell 2. The helmet 1 is then positioned with the aid of internal straps 18 so that the wearer can see clearly through the visor 6. The image received by the thermal imaging camera 8 is processed by the image interpretation circuit 10 to produce a video signal. The video signal is transferred to the augmented reality viewer 16 which converts the video signal to a video image which is then displayed on the transparent member 17. As the transparent member 17 is at the eye level of the wearer the wearer simultaneously sees both the scene through the visor 6 and a superimposed video image. Similar systems are used in aircraft where they are termed ‘head up displays’. When the wearer turns his/her head the image received by the thermal imaging camera 8 changes and the video image displayed by the augmented reality viewer 16 is automatically updated.

Power is supplied to both the thermal imaging camera 8 and the augmented reality viewer 16 by a power supply 19 located in the rear of the helmet shell 2, behind the wearer's head.

Such a known helmet 1 is relatively heavy than normal due to the presence of the image interpretation circuit 10 and power supply 19 in the helmet shell 2. In addition it is relatively expensive.

Shown in FIG. 2 is a schematic view of a helmet 20 according to the invention. The helmet 20 comprises a helmet shell 2, a breathing apparatus face mask 5, a visor 6 and an augmented reality viewer 16 as previously described.

Extending from the helmet shell 2 is a power socket 21 connected to the augmented reality viewer 16. The power socket 21 is adapted to be connected to a portable power supply 22 remote from the helmet 1.

The portable power supply 22 is typically located at the waist or on the back of the wearer of the helmet 1. The power supply 22 is used to power the augmented reality viewer 16.

Also extending from the helmet shell 2 is a video socket 23 connected to the augmented reality viewer 16. Video signals received by this video socket 23 are displayed by the augmented reality viewer 16. The video socket 23 is connected to a hand held thermal imaging camera 24 so that in use images received by the thermal imaging camera 24 are displayed by the augmented reality viewer 16. The thermal imaging camera 24 is connected indirectly to the video socket 23 by a video relay unit 25 as shown. In an alternative embodiment the camera 24 is connected directly to the video socket 23.

The camera 24 is powered by its own power supply. In an alternative embodiment the camera 24 is powered by the power supply 22 used to power the augmented reality viewer 16.

Shown in FIG. 3 is a second embodiment of a helmet according to the invention. The helmet 30 comprises a helmet shell 2, a breathing apparatus face mask 5, a visor 6, an augmented reality viewer 16 and a power socket 21 extending from the helmet shell 2 as previously described.

The helmet 30 further comprises a thermal imaging sensor 9 which is connected to an external image interpretation circuit 10. The external image interpretation circuit 10 is adapted to generate a video signal in response to the signal received from the thermal imaging sensor 9.

The external image interpretation circuit 10 is connected to a video socket 23 which extends from the helmet shell 2. The video signal generated by the external image interpretation circuit 10 is received by the video socket 23 for display by the augmented reality viewer 16.

The thermal imaging sensor 9 and the augmented reality viewer 16 are powered by an external power supply 22. In an alternative embodiment the thermal imaging sensor 9 is powered by a separate power supply to the augmented reality viewer 16.

In a further embodiment of a helmet according to the invention (not shown) the image interpretation circuit 10 is located within the helmet. Low weight circuitry is used so as not to unduly increase the weight of the helmet.

Shown in cross section in FIG. 4 is a viewing system 40 according to a further aspect of the invention. The viewing system 40 is mounted on a helmet in combination with a breathing apparatus face mask 5. The viewing system 40 comprises a housing 41 having straps 42 for mounting the viewing system 40 on to either the wearer of the helmet 5. Located within the housing 41 is an augmented reality viewer 16 and a thermal imaging sensor 9. The thermal imaging sensor 9 and the augmented reality viewer 16 are both connected to an external power source 22 by sockets 42 which extend from the viewing system housing 41. The thermal imaging sensor 9 is connected to an external image interpretation circuit 10 which converts the signal from the thermal imaging sensor 9 to a video signal. The video signal is then transmitted from the image interpretation circuit 10 to the augmented reality viewer 16 via a video socket 23 extending from the face mask housing 41.

In use the breathing apparatus face mask 5 and the viewing system 40 are fitted to the face in conjunction with a fire fighting helmet. This combination is used in conditions where the ability to breathe and to see is impaired. If visibility improves the viewing system 40 can be removed and the breathing apparatus face mask 5 and the helmet used as normal.

In a further embodiment of the viewing system according to the invention (not shown) the image interpretation circuit 10 is located withing the housing 41. Low weight circuitry is used so as not to unduly increase the weight of the viewing system.

1. A helmet comprising
a helmet shell;
a breathing apparatus face mask for connection to a breathing apparatus and a visor, the breathing apparatus face mask and the visor being connected to the helmet shell;
an augmented reality viewer positioned within the helmet shell and adapted to receive a video signal and to display the signal as a video image, the video image being arranged such that it can be viewed by a wearer of the helmet;
the helmet further comprising a power socket adapted to be connected to an external power source for the augmented reality viewer.

2. A helmet as claimed in claim 1, wherein the video image displayed by the augmented reality viewer is arranged such that it can be viewed by the wearer of the helmet whilst looking through the visor.

3. A helmet as claimed in claim 1, wherein the augmented reality viewer is located behind the breathing apparatus face mask.

4. A helmet as claimed in claim 1, wherein the helmet further comprises a video socket adapted to receive a video signal from an external source for display by the augmented reality viewer.

5. A helmet as claimed in claim 4, wherein the external video source comprises a thermal imaging camera.

6. A helmet as claimed in claim 4, wherein the helmet further comprises a thermal imaging sensor, the thermal imaging sensor being adapted to be connected to an external image interpretation circuit;
the image interpretation circuit being adapted to generate a video signal in response to the signal received from the thermal imaging sensor; and,
the video socket being adapted to receive the video signal generated by the external image interpretation circuit for display by the augmented reality viewer.

7. A helmet as claimed in claim 1, wherein the helmet further comprises a thermal imaging sensor, the thermal imaging sensor being connected to an integrated image interpretation circuit;
the image interpretation circuit being adapted to generate a video signal in response to the signal received from the thermal imaging sensor; and,
the augmented reality viewer being adapted to receive the video signal from the image interpretation circuit for display.

8. A viewing system for mounting on a breathing apparatus face mask, the viewing system comprising
an augmented reality viewer adapted to receive a video signal and to display the signal as a video image, the video image being arranged such that it can be viewed by a wearer of the face mask; and,
a power socket adapted to be connected to an external source of power for the augmented reality viewer.

9. A viewing system as claimed in claim 8, further comprising a thermal imaging sensor and integrated image interpretation circuit, the image interpretation circuit being adapted to generate a video signal in response to the signal received from the thermal imaging sensor;
the augmented reality viewer being adapted to receive the video image generated by the image interpretation circuit for display.

10. A viewing system as claimed in claim 8 further comprising a thermal imaging sensor adapted to be connected to an external image interpretation circuit, the external image interpretation circuit being adapted to generate a video signal in response to the signal received from the thermal imaging sensor;
the augmented reality viewer being adapted to receive the video image generated by the image interpretation circuit for display.
